(19) United States
(12) Patent Application Publication
(10) Pub. No.: US 2026/0083507 A1
(43) Pub. Date: Mar. 26, 2026

(54) PATIENT-SPECIFIC SIMULATION DEVICE AND METHOD FOR PLANNING TRANSCATHETER AORTIC VALVE IMPLANTATION

(71) Applicants: THE CATHOLIC UNIVERSITY OF KOREA INDUSTRY-ACADEMIC COOPERATION FOUNDATION, Seoul (KR); FOUNDATION FOR RESEARCH AND BUSINESS, SEOUL NATIONAL UNIVERSITY OF SCIENCE AND TECHNOLOGY, Seoul (KR)

(72) Inventors: Kwan Yong LEE, Seoul (KR); Hyoung Gwon CHOI, Seoul (KR); Truong Sang HA, Hanoi (VN); Kiyuk CHANG, Seoul (KR)

(73) Assignees: THE CATHOLIC UNIVERSITY OF KOREA INDUSTRY-ACADEMIC COOPERATION FOUNDATION, Seoul (KR); FOUNDATION FOR RESEARCH AND BUSINESS, SEOUL NATIONAL UNIVERSITY OF SCIENCE AND TECHNOLOGY, Seoul (KR)

(21) Appl. No.: 19/338,912

(22) Filed: Sep. 24, 2025

(30) Foreign Application Priority Data
Sep. 25, 2024 (KR) 10-2024-0130070

(51) Int. Cl. A61B 34/10; G06T 17/20; G16H 50/50

(52) U.S. Cl. CPC A61B 34/10; G06T 17/20; G16H 50/50; A61B 2034/105; A61B 2034/107; G06T 2210/24; G06T 2210/41

(57) ABSTRACT

Provided are an artificial intelligence AI-based simulation device and method for supporting procedure planning in transcatheter aortic valve implantation. The device receives patient-specific medical images, such as computed tomography data, and generates a three-dimensional (3D) model of the aortic root and native valve. An artificial valve is virtually inserted into the 3D model, and the virtual implantation model is automatically converted into mesh data suitable for Computational Fluid Dynamics (CFD) and Fluid-Structure Interaction (FSI) analysis. The processor performs CFD/FSI simulations to evaluate hemodynamic changes, structural stresses, and potential device-tissue interactions. Based on the analysis, the system outputs predicted risks of procedure-related complications (including coronary obstruction, annular rupture, paravalvular leakage, and valve deformation) and provides guideline data for selecting valve size, implantation depth, and insertion angle. The disclosed device and method enable accurate, patient-specific prediction and visualization, thereby assisting clinicians in establishing safe and optimized TAVI procedure plans.

### CROSS-REFERENCE TO RELATED APPLICATIONS

A claim for priority under 35 U.S.C. § 119 is made to Korean Patent Application No. 10-2024-0130070 filed on Sep. 25, 2024 in the Korean Intellectual Property Office, the entire contents of which are hereby incorporated by reference.

### BACKGROUND

Embodiments of the present disclosure described herein relate to a simulation device for simulating the optimal intervention method of transcatheter aortic valve implantation, and a method thereof.

Heart valve stenosis refers to a disease in which the valves between the ventricles and the aorta or between the atria and the ventricles narrow, thereby impairing the flow of blood from the heart to the rest of the body. In particular, about half of patients with aortic valve stenosis, where the aortic valve between the left ventricle and the aorta becomes narrowed, have a high risk of dying within two years when they do not undergo surgery. The heart valve stenosis is most common in people over the age of 65, which may be a major problem in worldwide aging society.

Severe aortic valve stenosis has been difficult to treat the patients surgically, especially when patients are old accompanying co-morbidities. However, since the world's first successful transcatheter aortic valve implantation (TAVI) in France in 2002, it has been actively performed worldwide, and several clinical studies have shown superior therapeutic effects in patients with high surgical risk.

In the meantime, with the increasing number of TAVI procedures, a variety of procedure-related complications have also been identified, many of which may be prevented when the target and non-target organ changes before and after a device is inserted may be predicted during the procedure.

Therefore, there is a need for a simulator that may accurately predict the changes before and after the device is inserted, based on the analysis of the patient's medical image data before the procedure for more successful procedure planning.

### SUMMARY

Embodiments of the present disclosure provide a simulation device that assists the establishment of safe and practical procedure plan through accurate predictions considering a patient's valve and aortic structural features prior to the TAVI procedure, and a method thereof.

Problems to be solved by the present disclosure are not limited to the problems mentioned above, and other problems not mentioned will be apparent by those skilled in the art from the following description.

According to an embodiment, a simulation device for simulating an optimal intervention method of transcatheter aortic valve implantation includes a communication module that receives a medical image of a patient with aortic valve stenosis, a memory that stores at least one process for performing simulation, a display that displays screen information, and a processor that performs the simulation according to the process. The processor generates a model of a three-dimensional (3D) aortic valve based on the medical image, generates a virtual implantation model in which an artificial valve is inserted into the 3D aortic valve, converts the virtual implantation model into mesh data capable of being used to analyze Computational Fluid Dynamics (CFD) and Fluid-Structure Interaction (FSI), analyzes the CFD and the FSI based on the converted mesh data, and controls the display such that the analysis result is output.

In an embodiment, the processor may generate at least one of optimal intervention guideline data and risk of complications according to at least one of a size, a location, and an angle of the artificial valve based on the analysis result, and may control the display such that the generated data is output.

In an embodiment, the present disclosure may further include an input module that receives artificial valve information from a user. The processor may generate a virtual implantation model, in which the artificial valve is inserted into the 3D aortic valve, based on the artificial valve information entered by the user.

In an embodiment, the processor may control the display such that the 3D aortic valve model is output, and may receive the artificial valve information while the 3D aortic valve model is output.

In an embodiment, the processor may control the display such that a guideline is displayed while overlapping the 3D aortic valve model.

In an embodiment, the processor may generate optimal artificial valve information based on the 3D aortic valve model, and may control the display such that the generated optimal artificial valve information is output.

In an embodiment, the processor may extract valve information of the patient with aortic valve stenosis from the 3D aortic valve model, and may generate the optimal artificial valve information based on the extracted valve information.

According to an embodiment, a method of simulating an optimal intervention method of transcatheter aortic valve implantation includes receiving, by a communication module, a medical image of a patient with aortic valve stenosis, generating, by a processor, a model of a 3D aortic valve based on the medical image, generating, by the processor, a virtual implantation model in which an artificial valve is inserted into the 3D aortic valve, converting, by the processor, the virtual implantation model into mesh data capable of being used to analyze CFD and FSI, analyzing, by the processor, the CFD and the FSI based on the converted mesh data, and outputting, by a display, the analysis result.

Besides, a computer program stored in a computer-readable recording medium for implementing the present disclosure may be further provided.

In addition, a computer-readable recording medium for recording a computer program for implementing the present disclosure may be further provided.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is an overall system diagram, according to an embodiment of the present disclosure;

FIG. 2 is a block diagram of a server included in a simulation device, according to an embodiment of the present disclosure;

FIG. 3 is a block diagram of a terminal included in a simulation device, according to an embodiment of the present disclosure;

FIG. 4 is a flowchart showing a simulation method, according to an embodiment of the present disclosure;

FIGS. 5 and 6 are example diagrams showing a 3D aortic valve model, according to an embodiment of the present disclosure;

FIG. 7 is an example diagram showing a model of fusing a 3D aortic valve and an artificial valve, according to an embodiment of the present disclosure;

FIG. 8 is an example diagram for describing a mesh data conversion method, according to an embodiment of the present disclosure;

FIG. 9 is an example diagram showing a model converting a 3D model of the present disclosure into mesh data;

FIG. 10 is an example diagram for interpreting the interaction between an artificial valve of the present disclosure and a blood flow;

FIGS. 11A to 11D are virtual simulator models using CFD and FSI analysis, according to an embodiment of the present disclosure;

FIG. 12 is an example diagram for describing a FSI analysis method, according to an embodiment of the present disclosure; and

FIG. 13 is an example diagram for describing a clinical application method, according to an embodiment of the present disclosure.

### DETAILED DESCRIPTION

The same reference numerals denote the same elements throughout the present disclosure. The present disclosure does not describe all elements of embodiments. Well-known content in a technical field, to which the present disclosure belongs, or redundant content in which embodiments are the same as one another will be omitted. A term such as ‘unit, module, member, or block’ used in the specification may be implemented with software or hardware. According to embodiments, a plurality of ‘units, modules, members, or blocks’ may be implemented with one component, or a single ‘unit, module, member, or block’ may include a plurality of components.

Throughout this specification, when it is supposed that a portion is “connected” to another portion, this includes not only a direct connection, but also an indirect connection. The indirect connection includes being connected through a wireless communication network.

Furthermore, when a portion “comprises” a component, it will be understood that it may further include another component, without excluding other components unless specifically stated otherwise.

Throughout this specification, when it is supposed that a member is located on another member “on”, this includes not only the case where one member is in contact with another member but also the case where another member is present between two other members.

Terms such as ‘first’, ‘second’, and the like are used to distinguish one component from another component, and thus the component is not limited by the terms described above.

Unless there are obvious exceptions in the context, a singular form includes a plural form.

In each step, an identification code is used for convenience of description. The identification code does not describe the order of each step. Unless the context clearly states a specific order, each step may be performed differently from the specified order.

Hereinafter, operating principles and embodiments of the present disclosure will be described with reference to the accompanying drawings.

In this specification, a ‘device according to an embodiment of the present disclosure’ includes all various devices capable of providing results to a user by performing arithmetic processing. For example, the device according to an embodiment of the present disclosure may include all of a computer, a server device, and a portable terminal, or may be in any one form.

Here, for example, the computer may include a notebook computer, a desktop computer, a laptop computer, a tablet PC, a slate PC, and the like, which are equipped with a web browser.

The server device may be a server that processes information by communicating with an external device and may include an application server, a computing server, a database server, a file server, a game server, a mail server, a proxy server, and a web server.

For example, the portable terminal may be a wireless communication device that guarantees portability and mobility, and may include all kinds of handheld-based wireless communication devices such as a smartphone, a personal communication system (PCS), a global system for mobile communication (GSM), a personal digital cellular (PDC), a personal handyphone system (PHS), a personal digital assistant (PDA), International Mobile Telecommunication (IMT)-2000, a code division multiple access (CDMA)-2000, W-Code Division Multiple Access (W-CDMA), and Wireless Broadband Internet (WiBro) terminal, and a wearable device such as a timepiece, a ring, a bracelet, an anklet, a necklace, glasses, a contact lens, or a head-mounted device (HMD).

A simulation device according to an embodiment of the present disclosure may be implemented by at least one of a server and a terminal. In particular, the device according to an embodiment of the present disclosure may be implemented by either a server or a terminal, or may be implemented as a system through data transmission/reception between the server and the terminal.

Hereinafter, the simulation device according to an embodiment of the present disclosure is described.

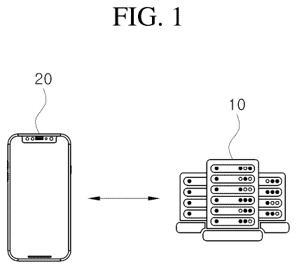

Referring to FIG. 1, a device according to an embodiment of the present disclosure may include a server 10 and a terminal 20.

The server 10 may be connected to the terminal 20 over a network, and may receive data required for diagnosis from the terminal 20, and then transmit the diagnosis results to the terminal 20.

In the meantime, the terminal 20 is not limited to the above-described portable terminal. For example, it is obvious to those skilled in the art that the terminal 20 may include a processor-equipped notebook, desktop, laptop, tablet PC, slate PC, or the like.

As described above, a simulation device according to an embodiment of the present disclosure may be implemented through data transmission/reception between the server 10 and the terminal 20.

Hereinafter, the server 10 and the terminal 20 for implementing the simulation device according to an embodiment of the present disclosure are described.

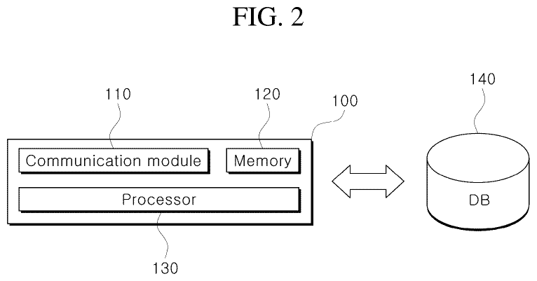

FIG. 2 is a block diagram of a server included in a simulation device, according to an embodiment of the present disclosure.

A server 100 according to an embodiment of the present disclosure may include at least one of a communication module 110, a memory 120, and a processor 130.

The communication module 110 may communicate with at least one of a terminal, external storage (e.g., a database 140), an external server, and a cloud server.

In the meantime, the external server or the cloud server may be configured to perform at least part of the role of the processor 130. In other words, data processing or data operations may be performed by an external server or a cloud server, and the present disclosure does not place any special restrictions on this method.

In the meantime, the communication module 110 may support various communication methods according to the communication standards of a communicating target (e.g., an electronic device, an external server, a device, etc.).

For example, the communication module 110 may be configured to communicate with a communication target by using at least one of wireless LAN (WLAN), Wireless-Fidelity (Wi-Fi), Wi-Fi Direct, digital living network alliance (DLNA), wireless broadband (WiBro), world interoperability for microwave access (WiMAX), high speed downlink packet access (HSDPA), high speed uplink packet access (HSUPA), long term evolution (LTE), long term evolution-advanced (LTE-A), 5th Generation (5G) Mobile Telecommunication, Bluetooth™, Radio Frequency Identification (RFID), Infrared Data Association (IrDA), Ultra-Wideband (UWB), ZigBee, Near Field Communication (NFC), and Wireless Universal Serial Bus (Wireless USB) technologies.

Next, the memory 120 may be configured to store various pieces of information related to the present disclosure. In an embodiment of the present disclosure, the memory 120 may be provided in the device itself according to an embodiment of the present disclosure. Unlike the above description, at least part of the memory 120 may refer to at least one of the database (DB) 140 and cloud storage (or a cloud server). In other words, it may be understood that the memory 120 is sufficient as a space for storing information necessary for the device and the method according to an embodiment of the present disclosure, and there are no restrictions on physical space. Accordingly, hereinafter, the memory 120, the database 140, the external storage, and the cloud storage (or the cloud server) may not be separately distinguished from each other, and may all be referred to as the “memory 120”.

Next, the processor 130 may be configured to control the overall operations of the device related to the present disclosure. The processor 130 may process a signal, data, information, or the like, which is input or output through the above-described components, or may provide or process information or functions suitable for user.

The processor 130 may include at least one central processing unit (CPU) and may perform functions according to an embodiment of the present disclosure.

At least one component may be added or deleted to correspond to the performance of the components illustrated in FIG. 2. Furthermore, it will be easily understood by those skilled in the art that mutual locations of the components may be changed to correspond to the performance or structure of the device.

Hereinafter, the terminal included in the device according to an embodiment of the present disclosure will be described in detail.

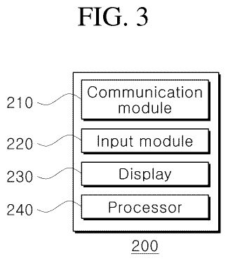

FIG. 3 is a block diagram of a terminal included in a device, according to an embodiment of the present disclosure.

Referring to FIG. 3, a terminal 200 according to an embodiment of the present disclosure may include a communication module 210, an input module 220, a display 230, and a processor 240. The components shown in FIG. 3 are not essential in implementing a device according to an embodiment of the present disclosure. The terminal described herein may have more or fewer components than those listed above.

Among the components, the communication module 210 may include one or more components capable of communicating with an external device, and may include, for example, at least one of a broadcast reception module, a wired communication module, a wireless communication module, a short-range communication module, and a location information module.

Here, in addition to various wired communication modules such as a Local Area Network (LAN) module, a Wide Area Network (WAN) module, or a Value Added Network (VAN) module, the wired communication module may include a variety of cable communication modules such as Universal Serial Bus (USB), High Definition Multimedia Interface (HDMI), Digital Visual Interface (DVI), recommended standard 1302 (RS-1302), power line communication, or plain old telephone service (POTS).

Here, the wireless communication module may include a wireless communication module for supporting various wireless communication methods such as Global System for Mobile (GSM) communication, Code Division Multiple Access (CDMA), Wideband Code Division Multiple Access (WCDMA), Universal Mobile Telecommunication System (UMTS), Time Division Multiple Access (TDMA), Long Term Evolution (LTE), 4G, 5G, and 6G in addition to a Wi-Fi module and Wireless broadband module.

The input module 220 may be used to enter image information (or signal), audio information (or signal), data, or information entered by a user. The input module 220 may include at least one of at least one camera, at least one microphone, and a user input unit. Speech data or image data collected by the input module may be analyzed and processed as a control command of a user.

The output module 230 may include at least one of a display, a sound output unit, a haptic module, and an optical output unit, which are used to generate an output associated with visual, auditory, or tactile sensation. The display may have a mutual layer structure with a touch sensor or may be integrally formed with the touch sensor, thereby implementing the touch screen. Such the touch screen may provide an output interface between the present device and a user as well as operating as a user input unit that provides an input interface between the present device and the user.

The display displays (outputs) information processed by the present device. For example, the display may display execution screen information of an application program (e.g., an application) running on the present device, or a user interface (UI) or graphical user interface (GUI) information according to such the execution screen information.

In addition to the components described above, the terminal described above may further include an interface unit and a memory.

The interface unit serves as a passage for various types of external devices connected to the present device. The interface unit may include at least one of a wired/wireless headset port, an external charger port, a wired/wireless data port, a memory card port, a port for connecting a device equipped with a subscriber identification module (SIM), an audio input/output (I/O) port, a video I/O port, and an earphone port. In the present device, appropriate control related to an external device connected to the interface unit may be performed.

The memory may store data for supporting various functions of the present device, and a program for operations of the processor, may store pieces of input/output data (e.g., music files, still images, videos, and the like), and may store a plurality of application programs (or applications) running on the present device, pieces of data for operations of the present device, and instructions. At least part of the application programs may be downloaded from an external server through wireless communication.

The memory may include the type of a storage medium of at least one of a flash memory type, a hard disk type, a Solid State Disk (SSD) type, a Silicon Disk Drive (SDD) type, a multimedia card micro type, a memory of a card type (e.g., SD memory, XD memory, or the like), a random access memory (RAM), a static random access memory (SRAM), a read-only memory (ROM), an electrically erasable programmable read-only memory (EEPROM), a programmable read-only memory (PROM), a magnetic memory, a magnetic disk, and an optical disc. Furthermore, the memory may be separate from the present device, but may be a database connected by wire or wirelessly.

In the meantime, the above-described terminal includes the processor 240. The processor may be implemented with a memory that stores data regarding an algorithm for controlling operations of components within the present device, or a program for implementing the algorithm, and at least one processor (not shown) that performs the above-described operations by using the data stored in the memory. At this time, the memory and the processor may be implemented as separate chips. Alternatively, the memory and the processor may be implemented as a single chip.

Meanwhile, the processor included in at least one of the server and the terminal may include an artificial intelligence module for implementing a simulation device to be described later. An artificial intelligence model training method to be described below is described as being implemented by the operation of the module, but the performance of an operation at each step to be described below does not necessarily have to be performed by the module.

Furthermore, to implement various embodiments of the present disclosure to be described below in the present device, the processor may control one of the components described above or the combination of the components.

In the meantime, at least one component may be added or deleted to correspond to the performance of the components illustrated in FIGS. 1 to 3. Furthermore, it will be easily understood by those skilled in the art that mutual locations of the components may be changed to correspond to the performance or structure of the device.

Hereinafter, the artificial intelligence described in the present disclosure is described in detail.

Functions related to the artificial intelligence according to an embodiment of the present disclosure are operated through a processor and a memory, which are installed in the server and the terminal. The processor may consist of one or more processors. In this case, the one or more processors may be a general-purpose processor (e.g., a CPU, an AP, or a digital signal processor (DSP)), a graphics-dedicated processor (e.g., a GPU or a vision processing unit (VPU)), or an artificial intelligence (AI)-dedicated processor (e.g., an NPU). Under control of the one or more processors, input data may be processed depending on an AI model, or a predefined operating rule stored in the memory. Alternatively, when the one or more processors are AI-dedicated processors, the AI-dedicated processor may be designed with a hardware structure specialized for processing a specific AI model.

The predefined operating rule or the artificial intelligence model is created through learning. Here, being created through learning means creating the predefined operating rule or the artificial intelligence model configured to perform desired features (or purposes) as a basic artificial intelligence model is learned by using pieces of learning data by a learning algorithm. This learning may be performed by a device itself, on which the artificial intelligence according to an embodiment of the present disclosure is performed, or may be performed through a separate server and/or a system. For example, the learning algorithm may include supervised learning, unsupervised learning, semi-supervised learning, or reinforcement learning, but may not be limited to the above example.

An artificial intelligence model may be composed of a plurality of neural network layers. The plurality of neural network layers respectively have a plurality of weight values, and each of the plurality of neural network layers performs neural network calculation through calculations between the calculation result of the previous layer and the plurality of weight values. The plurality of weight values of the plurality of neural network layers may be optimized by the learning result of the artificial intelligence model. For example, during a learning process, the plurality of weight values may be updated such that a loss value or a cost value obtained from the artificial intelligence model is reduced or minimized. The artificial neural network may include a deep neural network (DNN). The artificial neural network may be, for example, a convolutional neural network (CNN), a deep neural network (DNN), a recurrent neural network (RNN), a restricted Boltzmann machine (RBM), a deep belief network (DBN), a bidirectional recurrent deep neural network (BRDNN), or a deep Q-network, but is not limited to the above-described example.

According to an embodiment of the present disclosure, a processor may implement artificial intelligence. The artificial intelligence may refer to an artificial neural network-based machine learning method that allows a machine to perform learning by simulating human biological neurons. The methodology of artificial intelligence may be classified depending on a learning method as supervised learning, in which a solution (output data) to a problem (input data) is determined by providing input data and output data together as training data, unsupervised learning, in which only input data is provided without output data, and thus the solution (output data) to the problem (input data) is not determined, and reinforcement learning, in which a reward is given from an external environment whenever an action is taken in a current state, and thus learning progresses to maximize this reward. Moreover, the methodology of artificial intelligence may also be categorized depending on architecture, which is the structure of the learning model. The architecture of deep learning technology widely used may be categorized into convolutional neural networks (CNN), recurrent neural networks (RNN), transformers, and generative adversarial networks (GAN).

Each of the present device and the system may include an artificial intelligence model. The artificial intelligence model may be a single artificial intelligence model or may be implemented as a plurality of artificial intelligence models. The artificial intelligence model may be composed of neural networks (or artificial neural networks) and may include a statistical learning algorithm that mimics biological neurons in machine learning and cognitive science. The neural network may refer to a model as a whole having the ability to solve problems as artificial neurons (nodes), which form a network by connecting synapses, changes the strength of their synaptic connections through learning. Neurons in the neural network may include the combination of weight values or biases. The neural network may include one or more layers consisting of one or more neurons or nodes. For example, the present device may include an input layer, a hidden layer, and an output layer. The neural network constituting the present device may infer the result (output) to be predicted from an arbitrary input by changing a weight value of a neuron through learning.

The processor may create a neural network, may train or learn a neural network, or may perform operations based on received input data, and then may generate an information signal or may retrain the neural network based on the performed results. Models of a neural network may include various types of models such as a convolution neural network (CNN) (e.g., GoogleNet, AlexNet, or VGG Network), a region with convolution neural network (R-CNN), a region proposal network (RPN), a recurrent neural network (RNN), a stacking-based deep neural network (S-DNN), a state-space dynamic neural network (S-SDNN), a deconvolution network, a deep belief network (DBN), a restricted Boltzman machine (RBM), a fully convolutional network, a long short-term memory (LSTM) Network, and a classification network, but is not limited thereto. The processor may include one or more processors for performing computations according to the models of the neural network. For example, the neural network may include a deep neural network.

It will be understood by those skilled in the art that a neural network may include any neural network, but is not limited to a convolutional neural network (CNN), a recurrent neural network (RNN), a perceptron, a multilayer perceptron, a feed forward (FF), a radial basis network (RBF), a deep feed forward (DFF), a long short term memory (LSTM), a gated recurrent unit (GRU), an auto encoder (AE), a variational auto encoder (VAE), a denoising auto encoder (DAE), a sparse auto encoder (SAE), a Markov chain (MC), a Hopfield network (HN), a Boltzmann machine (BM), a restricted Boltzmann machine (RBM), a deep belief network (DBN), a deep convolutional network (DCN), a deconvolutional network (DN), a deep convolutional inverse graphics network (DCIGN), a generative adversarial network (GAN), a liquid state machine (LSM), an extreme learning machine (ELM), an echo state network (ESN), a deep residual network (DRN), a differentiable neural computer (DNC), a neural turning machine (NTM), a capsule network (CN), a Kohonen network (KN), and an attention network (AN).

According to an embodiment of the present disclosure, the processor may use various artificial intelligence structures and algorithms such as a convolution neural network (CNN) (e.g., GoogleNet, AlexNet, or VGG Network), a region with convolution neural network (R-CNN), a region proposal network (RPN), a recurrent neural network (RNN), a stacking-based deep neural network (S-DNN), a state-space dynamic neural network (S-SDNN), a deconvolution network, a deep belief network (DBN), a restricted Boltzmann machine (RBM), a fully convolutional network, a long short-term memory (LSTM) Network, a classification network, Generative Modeling, explainable AI, Continual AI, Representation Learning, AI for Material Design, algorithms for natural language processing (e.g., BERT, SP-BERT, MRC/QA, Text Analysis, Dialog System, GPT-3, and GPT-4), algorithms for vision processing (e.g., Visual Analytics, Visual Understanding, Video Synthesis, and ResNet), algorithms for data intelligence (e.g., Anomaly Detection, Prediction, Time-Series Forecasting, Optimization, Recommendation, and Data Creation), but is not limited thereto. Hereinafter, an embodiment of the present disclosure will be described in detail with reference to the accompanying drawings.

In an embodiment, the processor may perform auto-segmentation by using a segment anything model.

Hereinafter, the simulation method utilizing the above-described components is described in detail. The simulation method to be described below may be implemented through data transmission/reception between the server and the terminal described above, and some steps of the method may be performed by either the server or the terminal. However, it is obvious to those skilled in the art that the method to be described below is not limited thereto and may be performed independently by either the server or the terminal.

Hereinafter, a simulation method utilizing the components described in FIGS. 1 to 3 is described.

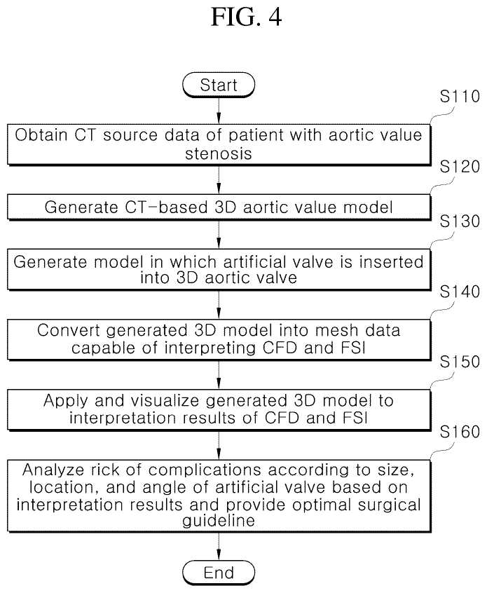

FIG. 4 is a flowchart showing a simulation method, according to an embodiment of the present disclosure.

First, an operation of receiving a medical image of a patient with aortic valve stenosis is performed (S110).

The medical image may be a CT image source data of the patient with aortic valve stenosis. For patients undergoing transcatheter aortic valve implantation (referred to as “TAVI”), computed tomography (CT) is performed rather than MRI for pre-procedure diagnosis and procedure planning. The present disclosure performs simulation by using the CT image.

Next, an operation of generating a three-dimensional (3D) aortic valve model based on the CT image is performed (S120).

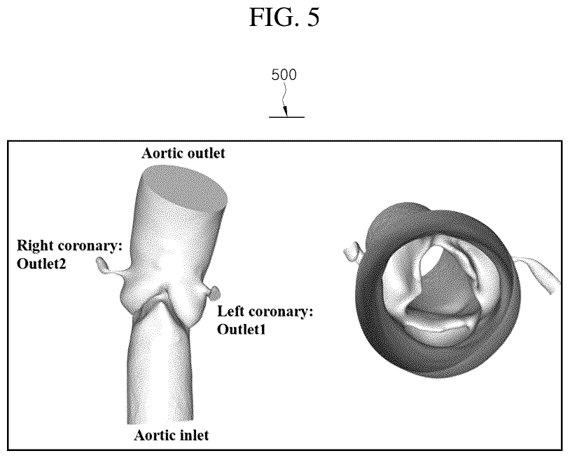

A processor generates the 3D aortic valve model by using CT image source data consisting of a plurality of two-dimensional (2D) images. For example, referring to FIG. 5, the thickness of an aorta, the shape of the aorta, the location of a valve, the number of valves, the shape of the valve, or the like may be identified through a 3D aortic valve model 500.

Here, the method of generating the 3D aortic valve model by using a 2D CT image is not particularly limited thereto. For example, any known 3D modeling technique may be utilized.

Next, an operation of generating a model in which an artificial valve is inserted into a 3D aortic valve is performed (S130).

Here, the processor may generate a virtual implantation model in which an artificial valve is inserted into the 3D aortic valve by using artificial valve information input from a user.

In an embodiment, the artificial valve information may include at least one of the size of the artificial valve to be inserted, the product name of the artificial valve, an insertion location of the artificial valve, and an insertion angle of the artificial valve.

The processor may generate a virtual implantation model based on the type and size of the artificial valve that a user wants to virtually insert, and the location and angle of the artificial valve that the user wants to insert.

In an embodiment, the processor may allow the display to display the 3D aortic valve model, and may receive the artificial valve information while the 3D aortic valve model is output. In other words, the present disclosure allows the user to enter the artificial valve information to be virtually inserted while the user views the 3D aortic valve model.

In an embodiment, the processor may control the display such that a guideline is displayed on the 3D aortic valve model while overlapping the 3D aortic valve model. The guideline may be a guideline that allows the artificial valve to be inserted correctly into an aorta. For example, the guideline may be a line that guides the central part of the aorta. The user may place the artificial valve such that the central part of the artificial valve is aligned with the central part of the aorta with reference to the guideline.

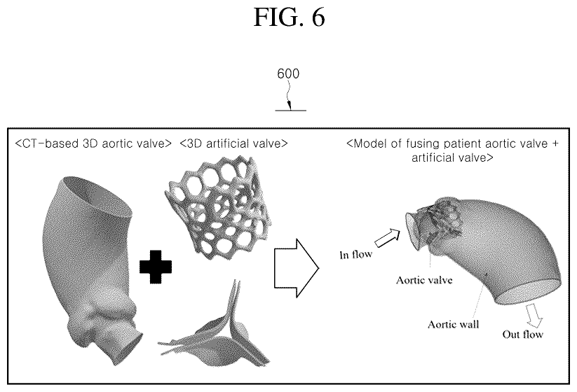

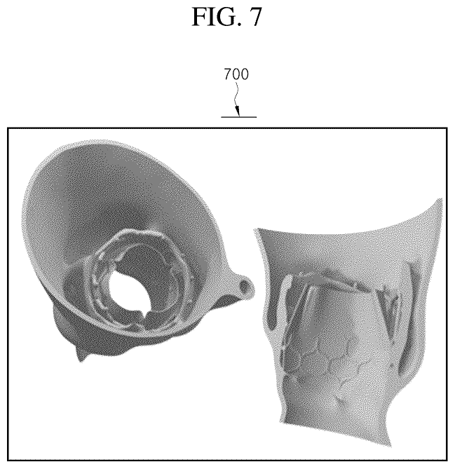

For example, referring to FIGS. 6 and 7, the present disclosure may generate virtual implantation models 600 and 700 in which an artificial valve is inserted into an aortic valve by using a CT-based 3D aortic valve model and the artificial valve information.

Next, an operation of converting the virtual implantation model into mesh data capable of Computational Fluid Dynamics (CFD) and Fluid-Structure Interaction (FSI) is performed (S140). Moreover, an operation of performing CFD and FSI on the generated virtual implantation model based on the converted mesh data, and outputting the analysis results is performed (S150).

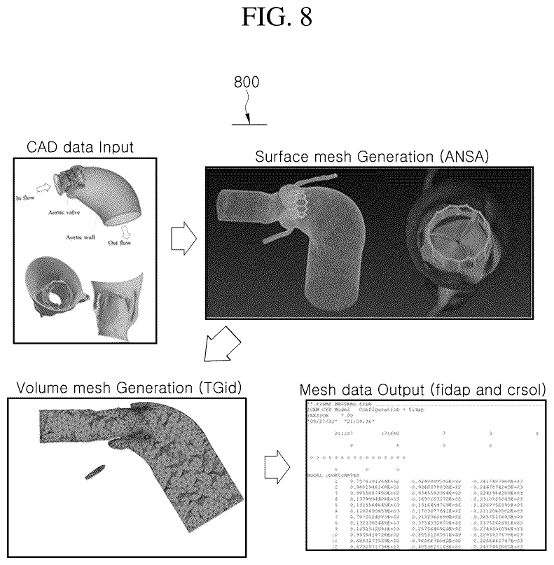

Referring to FIG. 8, a process 800 of converting the virtual implantation model into mesh data capable of CFD and FSI analysis may be performed in a method of generating a surface mesh (using ANSA) by using a virtual implantation model and then generating a volume mesh (using TGrid) to create final mesh data.

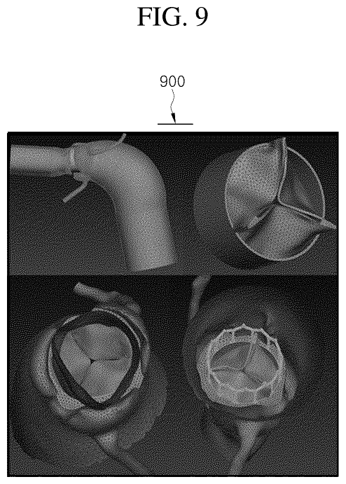

As a result, referring to FIG. 9, a model 900 that converts a 3D virtual implantation model into mesh data may be generated.

In an embodiment, an FSI analysis algorithm based on a ‘Semi-monolithic technique’ may be used to create a simulation analysis model before and after a procedure by using the FSI technique. This technique has very robust properties and fast convergence properties in the analysis of FSI problems where densities of solids and fluids are similar to each other, such as the interaction of blood vessels and blood flow.

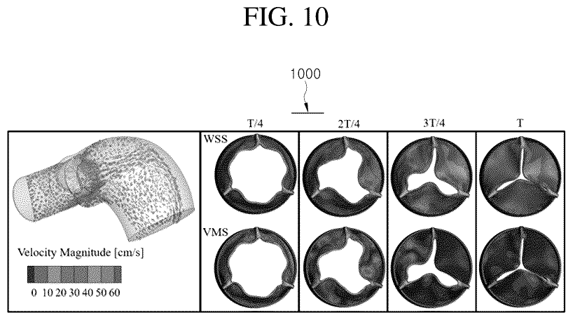

FIG. 10 shows a distribution 1000 of wall shear stress and von-Mises stress on a valve surface as the preliminary calculation result of interpreting the interaction between an artificial valve and a blood flow by using the developed code, and shows superior performance that is approximately 40 times faster than that of the commercial code ANSYS. The preliminary calculation code may take several days to perform computations by using a single core, but the analysis using multiple cores may provide analysis results in less than an hour. Re-meshing may be manually performed by a user and may generate a mesh in less than a minute.

Specifically, FIG. 10 visualizes the results obtained by performing FSI analysis, and the left drawing shows a blood flow at one moment of systole when an artificial valve opens. The upper right diagram shows a wall shear stress distribution on the valve surface changing over time, and the lower right diagram shows von-Mises stress, which is the structure stress acting on a valve and which changes over time.

Finally, an operation of generating and outputting at least one of optimal intervention guideline data and the risk of complications according to at least one of a size, a location, and an angle of an artificial valve based on the analysis results is performed (S160).

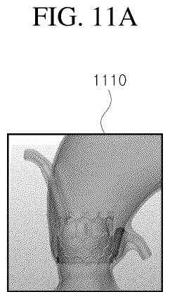

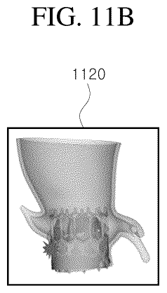

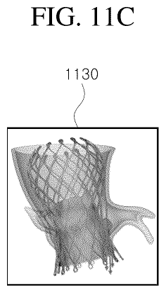

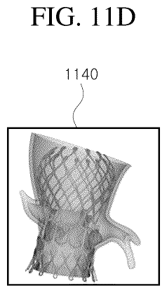

Referring to FIGS. 11A to 11D, it is possible to indicate locations 1110, 1120, 1130, and 1140 where complications are likely to occur in a TAVI virtual simulator using CFD and FSI analysis.

In particular, FIG. 11A shows that the entrance of the coronary artery is occluded while an artificial valve pushes against the front end of the existing valve (1110); FIG. 11B shows a blood vessel rupture when the artificial valve pushes against a calcified mass in the existing valve (1120); FIG. 11C shows the leakage phenomenon caused by the clearance around the valve after insertion of the artificial valve (1130); and FIG. 11D shows the process of determining the optimal size and shape of the artificial valve by changing and applying the type of artificial valve (1140).

To achieve clinically usable codes, in addition to the previously developed (1) “Multi-Grid” solution for unalignable grids to accelerate convergence of the pressure equation and (2) a technique for simulating nonlinear large deformations of solids and handling multiple material properties, (3) an automatic re-meshing function using the advancing front algorithm, and (4) a parallelized code based on a region partitioning technique to provide FSI analysis results in approximately one hour may be utilized in the present disclosure.

In the meantime, to visualize the interpretation results, commercial software such as TECPLOT or an open source code such as PARAVIEW may be utilized.

In summary, the simulation device and the simulation method according to an embodiment of the present disclosure may generate a 3D aortic valve model by smoothing and optimizing mesh data of the generated 3D model, may apply the CFD and FSI analysis results analyzed in the prior development process to simulation, and may analyze the risk of possible complications and the optimal TAVI procedure method through visualization so as to be provided to a practitioner.

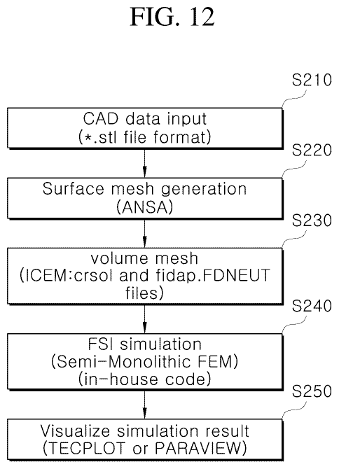

FIG. 12 is a visualization process of the results of simulating a model generated by using the CT of a patient before a procedure. The simulation result may be visualized (S250) through the process of entering a virtual model (S210), generating a surface mesh (S220), generating a volume mesh (S230), and FSI simulation (S240).

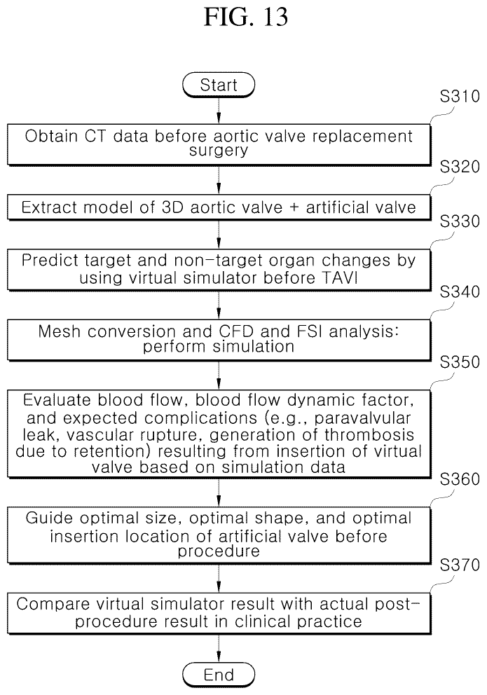

In the meantime, referring to FIG. 13, based on simulation methods S310 to S350 described above, optimal artificial valve information may be generated and output (S360) before TAVI surgery. Here, the optimal artificial valve information may include information about an optimal size, an optimal shape, and an optimal insertion location of an artificial valve.

In an embodiment, a processor may extract valve information of an aortic valve stenosis patient from the 3D aortic valve model and may generate the optimal artificial valve information based on the extracted valve information.

In the meantime, the optimal artificial valve information may be displayed when a user enters artificial valve information for generating a virtual model. In this way, the present disclosure may minimize the time, which is required for the user to perform a simulation, to obtain optimal treatment results.

Meanwhile, an operation of comparing the simulation results with the actual post-procedure results may be performed (S370). An artificial intelligence model that is trained by using the above-described comparison results and generates guide information that guides the optimal size, the optimal shape, and the optimal insertion location of an artificial valve by using the patient's CT image as input data may be implemented.

As described above, a simulation device and a method thereof according to an embodiment of the present disclosure may establish a TAVI procedure plan for minimizing complications by providing a risk level of possible complications according to a TAVI procedure plan and providing optimal artificial valve information suitable for a patient.

Moreover, the simulation device and the simulation method according to an embodiment of the present disclosure may be utilized for educational purposes in medical education and education institutions as well as for supporting the procedure planning of clinicians. In this way, the present disclosure may help improve the skills and practical training of medical professionals.

Meanwhile, the disclosed embodiments may be implemented in a form of a recording medium storing instructions executable by a computer. The instructions may be stored in a form of program codes, and, when executed by a processor, generate a program module to perform operations of the disclosed embodiments. The recording medium may be implemented as a computer-readable recording medium.

The computer-readable recording medium may include all kinds of recording media in which instructions capable of being decoded by a computer are stored. For example, there may be read only memory (ROM), random access memory (RAM), magnetic tape, magnetic disk, flash memory, optical data storage device, and the like.

Disclosed embodiments are described above with reference to the accompanying drawings. One ordinary skilled in the art to which the present disclosure belongs will understand that the present disclosure may be practiced in forms other than the disclosed embodiments without altering the technical ideas or essential features of the present disclosure. The disclosed embodiments are examples and should not be construed as limited thereto.

A simulation device and a method thereof according to an embodiment of the present disclosure may establish a TAVI procedure plan for minimizing complications by providing a risk level of possible complications according to a TAVI procedure plan and providing optimal artificial valve information suitable for a patient.

Effects of the present disclosure are not limited to the effects mentioned above, and other effects not mentioned will be apparent by those skilled in the art from the following description.

While the present disclosure has been described with reference to embodiments, it will be apparent to those skilled in the art that various changes and modifications may be made without departing from the spirit and scope of the present disclosure. Therefore, it should be understood that the above embodiments are not limiting, but illustrative.

What is claimed is:

1. A simulation device for simulating an optimal intervention method of transcatheter aortic valve implantation, the simulation device comprising:
a communication module configured to receive a medical image of a patient with aortic valve stenosis;
a memory configured to store at least one process for performing simulation;
a display configured to display screen information; and
a processor configured to perform the simulation according to the process,
wherein the processor is configured to:
generate a model of a three-dimensional (3D) aortic valve based on the medical image;
generate a virtual implantation model in which an artificial valve is inserted into the 3D aortic valve;
convert the virtual implantation model into mesh data capable of being used to analyze Computational Fluid Dynamics (CFD) and Fluid-Structure Interaction (FSI);
analyze the CFD and the FSI based on the converted mesh data; and
control the display such that the analysis result is output.

2. The simulation device of claim 1, wherein the processor is configured to:
generate at least one of optimal intervention guideline data and risk of complications according to at least one of a size, a location, and an angle of the artificial valve based on the analysis result; and
control the display such that the generated data is output.

3. The simulation device of claim 2, further comprising:
an input module configured to receive artificial valve information from a user,
wherein the processor is configured to:
generate the virtual implantation model, in which the artificial valve is inserted into the 3D aortic valve, based on the artificial valve information entered by the user.

4. The simulation device of claim 3, wherein the processor is configured to:
control the display such that the 3D aortic valve model is output; and
receive the artificial valve information while the 3D aortic valve model is output.

5. The simulation device of claim 4, wherein the processor is configured to:
control the display such that a guideline is displayed while overlapping the 3D aortic valve model.

6. The simulation device of claim 5, wherein the processor is configured to:
generate optimal artificial valve information based on the 3D aortic valve model; and
control the display such that the generated artificial valve information is output.

7. The simulation device of claim 6, wherein the processor is configured to:
extract valve information of the patient with aortic valve stenosis from the 3D aortic valve model; and
generate the artificial valve information based on the extracted valve information.

8. A method, which is performed by a device and which simulates an optimal intervention method of transcatheter aortic valve implantation, the method comprising:
receiving, by a communication module, a medical image of a patient with aortic valve stenosis;
generating, by a processor, a model of a 3D aortic valve based on the medical image;
generating, by the processor, a virtual implantation model in which an artificial valve is inserted into the 3D aortic valve;
converting, by the processor, the virtual implantation model into mesh data capable of being used to analyze CFD and FSI;
analyzing, by the processor, the CFD and the FSI based on the converted mesh data; and
outputting, by a display, the analysis result.

9. The method of claim 8, further comprising:
generating, by the processor, at least one of optimal intervention guideline data and risk of complications according to at least one of a size, a location, and an angle of the artificial valve based on the analysis result; and
outputting, by the display, the generated data.

10. The method of claim 9, further comprising:
receiving, by an input module, artificial valve information from a user,
wherein the generating of the virtual implantation model in which the artificial valve is inserted into the 3D aortic valve is performed based on the artificial valve information received from the user.

11. The method of claim 10, further comprising:
outputting, by the display, the 3D aortic valve model,
wherein the receiving, by the input module, of the artificial valve information from the user is performed while the 3D aortic valve model is output.

12. The method of claim 11, wherein the outputting, by the display, of the 3D aortic valve model includes:
displaying a guideline while the guideline overlaps the 3D aortic valve model.

13. The method of claim 12, further comprising:
generating, by the processor, optimal artificial valve information based on the 3D aortic valve model; and
outputting, by the display, the generated artificial valve information.

14. The method of claim 13, wherein the generating of the optimal artificial valve information based on the 3D aortic valve model includes:
extracting valve information of the patient with aortic valve stenosis from the 3D aortic valve model; and
generating the artificial valve information based on the extracted valve information.

15. A non-transitory computer-readable medium storing instructions that, when executed by a processor, cause the processor to perform a method of simulating a TAVI procedure, the method comprising:
receiving patient-specific medical image data of a patient with aortic valve stenosis;
generating a 3D aortic valve model based on the medical image data;
generating a virtual implantation model in which an artificial valve is virtually inserted into the 3D aortic valve model;
converting the virtual implantation model into mesh data suitable for CFD and FSI analysis:
analyzing the CFD and the FSI based on the mesh data; and
outputting, to a display, an analysis result including at least one of:
(i) predicted complication risks associated with the artificial valve implantation, or
(ii) intervention guideline information for selecting at least one of valve size, implantation depth, or insertion angle.
